(19) United States
(12) Patent Application Publication
(10) Pub. No.: US 2024/0139003 A1
(43) Pub. Date: May 2, 2024

(54) BIORESORBABLE STENT AND METHOD FOR MANUFACTURING THE SAME

(71) Applicant: INDUSTRY FOUNDATION OF CHONNAM NATIONAL UNIVERSITY, Gwangju (KR)

(72) Inventors: Myung Ho JEONG, Gwangju (KR); Dae Sung PARK, Gwangju (KR); Jae Un KIM, Gwangju (KR); Mun Ki KIM, Gwangju (KR); Doo Sun SIM, Gwangju (KR); Kyung Hoon CHO, Gwangju (KR); Dae Young HYUN, Gwangju (KR); Jun Kyu PARK, Gwangju (KR)

(21) Appl. No.: 18/559,765

(22) PCT Filed: May 13, 2022
(86) PCT No.: PCT/KR2022/006929

§ 371 Date: Dec. 18, 2023

(30) Foreign Application Priority Data
May 13, 2021 (KR) 10-2021-0062206

(51) Int. Cl. A61F 2/89; A61L 31/04; A61L 31/08; A61L 31/14; A61L 31/18

(52) U.S. Cl. CPC A61F 2/89; A61L 31/04; A61L 31/08; A61L 31/148; A61L 31/18; A61F 2210/0004; A61F 2240/001; A61F 2310/00005; A61F 2310/00389

(57) ABSTRACT

Provided is a bioresorbable stent including a stent substrate including a bioresorbable polymer and a contrast medium containing an iodine component, coated on the stent substrate. Since the stent according to the present invention is absorbed in and removed from the human body after a predetermined time, it has excellent biodegradability since it has improved radiopacity by iodine contrast medium coating, it has a high radiography contrast and is very efficient even when a procedure is performed with real time radiography, and since it has low foreshortening and high flexibility, radial force, and re-coil, it may be useful for insertion into a blood vessel having a small diameter, an acute occlusive lesion, an imminent occlusive lesion, and the like.

### TECHNICAL FIELD

The present invention relates to a bioresorbable stent and a method for manufacturing the same.

### BACKGROUND ART

A medical stent is a medical appliance which, when a blood vessel becomes narrower due to various diseases occurring in the human body to cause bad blood circulation, is placed inside the blood vessel to dilate the vessel.

Specifically, the stent is a medical appliance which, when a blood vessel becomes narrower due to various diseases occurring in the human body to cause bad blood circulation, is placed inside the blood vessel to dilate the vessel. There are various stent procedure methods, but the stent is mainly placed by balloon dilatation in which the stent is inserted into blood vessels such as the heart blood vessels, aorta, and cerebral blood vessels together with a balloon catheter, and expands tubular passages as the balloon inflates. Since a conventional stent expands outward along with the inflation of the balloon, it is required to have elasticity and ductility in order to expand to the original size of a vascular passage. That is, a stent is required to have ductility for insertion into a complex and curved passage during a procedure of inserting and fixing a balloon catheter to a target area and then inflating the balloon to dilate a narrowed area. In addition, after the procedure, the conditions such as elasticity for preventing deformation of a stent structure by a contraction force of a blood vessel (such as a heart blood vessel, an aorta, and a cerebral artery) tissue are required.

In addition, materials forming the stent are required to have excellent biochemical properties such as high biocompatibility and stability to the human body and chemical properties such as high corrosion resistance.

As an example, a stent including a diamond-like carbon thin film layer, a surface coating method thereof, and a surface coating device thereof are known in Korean Patent Laid-Open Publication No. 10-2010-0095942. The stent of the patent publication has a silicon-based buffer layer coated on a stent substrate and a diamond carbon thin film layer coated on the buffer layer, and has excellent physical properties of a diamond carbon such as a low friction coefficient and high corrosion resistance being applied.

However, since in the stent of the patent publication, the silicon-based buffer layer is formed between the stent substrate and the diamond carbon thin film layer, the stent has a relatively improved bonding force, but when stress is concentrated on a certain part by constantly applying a force by expansion, contraction, and the like to a stent structure, a problem that a surface coating layer is peeled off or cracks occur may not be avoided. In addition, since the silicon-based buffer layer used for improving a bonding force in the stent of the patent publication does not have better biocompatibility, a problem such as late thrombosis or induction of inflammation may arise after a stent procedure. In addition, since the silicon-based compound causes a change in a thin film layer by a rapid binding reaction with a specific material such as oxygen upon short-term exposure to air, a bonding force to a diamond-like carbon thin film layer is lowered and peeling and crack incidences are increased.

Therefore, a medical stent should have excellent biocompatibility properties as well as excellent physical properties such as friction coefficient, strength, ductility, and elasticity described above, and when stress is concentrated on a certain part by constantly applying force, a problem of surface coating layer peeling off or crack occurrence should be minimized.

### DISCLOSURE

### Technical Problem

An object of the present invention is to provide a bioresorbable stent which has excellent biocompatibility, high imaging resolution in radiography, and a structure showing appropriate physical properties for use in vascular insertion.

### Technical Solution

In one general aspect, a bioresorbable stent includes: a stent substrate including a bioresorbable polymer; and a contrast medium containing an iodine component, coated on the stent substrate.

In another general aspect, a method for manufacturing a bioresorbable stent includes: coating a contrast medium containing an iodine component on a stent substrate including a bioresorbable polymer.

In still another general aspect, a bioresorbable stent includes: a plurality of rings which are arranged to be spaced apart at a predetermined interval in the axial direction; and at least one bridge which is arranged between adjacent two rings of the plurality of rings and connects the adjacent two rings, wherein in each of the plurality of rings, a wave-shaped unit structure including a protrusion and a depression is arranged in a repeating manner along the circumferential direction, the unit structure includes the protrusion and the depression asymmetrically, and the bridge has a curvature.

### Advantageous Effects

The present invention relates to a bioresorbable stent including a stent substrate including a bioresorbable polymer and a contrast medium containing an iodine component, coated on the stent substrate. Since the stent according to the present invention is absorbed in and removed from the human body after a predetermined time, it has excellent biodegradability, since it has improved radiopacity by iodine contrast medium coating, it has a high radiography contrast and is very efficient even when a procedure is performed with real-time radiography, and since it has low foreshortening and high flexibility, radial force, and re-coil, it may be useful for insertion into a blood vessel having a small diameter, an acute occlusive lesion, an imminent occlusive lesion, and the like.

### DESCRIPTION OF DRAWINGS

FIG. 1 is a perspective view of a stent according to an exemplary embodiment of the present invention.

FIG. 2 is a development view of the stent according to an exemplary embodiment of the present invention.

FIG. 3 is a development view of a stent of the comparative example which is a commercial stent.

FIG. 4 is an enlarged view of a part of the stent of the comparative example which is a commercial stent.

FIGS. 5A and 5B are simple schematic diagrams for analyzing radial force in finite-element analysis of Experimental Example 1 of the present invention.

FIGS. 6A and 6B are drawings showing results of analyzing foreshortening in the finite-element analysis of Experimental Example 1 of the present invention.

FIG. 7 is a simple schematic diagram for crush resistance analysis in the finite-element analysis of Experimental Example 1 of the present invention.

FIG. 8 is a simple schematic diagram for flexibility analysis in the finite-element analysis of Experimental Example 1 of the present invention.

FIG. 9 is a drawing showing a radial force analysis experiment photograph and the results in a mechanical test of Experimental Example 2 of the present invention.

FIG. 10 is a drawing showing a foreshortening analysis experiment photograph and the results in the mechanical test of Experimental Example 2 of the present invention.

FIG. 11 is a drawing showing a flexibility analysis experiment photograph and the results of the mechanical test of Experimental Example 2 of the present invention.

FIG. 12 is a drawing showing a re-coil analysis experiment photograph and the results of the mechanical test of Experimental Example 2 of the present invention.

FIGS. 13A and 13B are photographs observed by a scanning electron microscope before and after coating a contrast medium of step 2 of the example of the present invention.

FIG. 14 is a drawing showing radiolucency experiment results of BMS (metal stent), the comparative example (commercial stent), BRS (untreated bioresorbable stent, stent manufactured in step 1 of the example), and With CM-BRS (bioresorbable stent coated with a contrast medium, stent manufactured in step 2 of the example), in which above is a photograph of a real stent and below is a photograph taken by X-ray.

### BEST MODE

Hereinafter, the present invention will be described in detail.

Meanwhile, the exemplary embodiments of the present invention may be modified in many different forms and the scope of the invention is not be limited to the exemplary embodiments set forth herein. In addition, the exemplary embodiments of the present invention are provided in order to explain the present invention more completely to those with ordinary skill in the art. Furthermore, throughout the specification, unless explicitly described to the contrary, “comprising” any constituent elements will be understood to imply further inclusion of other constituent elements rather than exclusion of other constituent elements.

An aspect of the present invention provides a bioresorbable stent (BRS) including:
a stent substrate including a bioresorbable polymer; and
a contrast medium containing an iodine component, coated on the stent substrate.

The bioresorbable polymer may be polylactic acid, polylactide, polyglycolide, polycaprolactone, polylactide-co-glycolide, polylactide-co-caprolactone, polyglycolide-co-caprolactone, polydioxanone, polytrimethylene carbonate, polyglycolide-co-dioxanone, polyamide ester, polypeptide, polyorthoester-based, polymaleic acid, polyphosphagen, polyanhydride, polysebacic anhydride, polyhydroxyalkanoate, polyhydroxybutylate, or polycyanoacrylate. When a stent manufactured using poly-L-lactic acid (PLLA) which is the bioresorbable polymer by an exemplary embodiment of the present invention is inserted into the human body, the stent was confirmed to be absorbed well and removed from the human body.

The contrast medium containing an iodine component may increase radiopacity, and the contrast medium containing an iodine component may be, for example, a contrast medium selected from the group consisting of Iopromide, Iopamidol, Iohexol, Iodixanol, Amidtrizoic acid, Iokisagulic acid, Ioxylan, Iotalamic acid, Isotroxylic acid meglumine, Iotrolan, Iopanoic acid, Iomeprol, sodium iofordate, Iodamide, iodochisamic acid, and combinations thereof. It is preferred that the contrast medium containing an iodine component is coated on the stent substrate by an electrospinning technique.

Another aspect of the present invention provides a method for manufacturing a bioresorbable stent including:
coating a contrast medium containing an iodine component on a stent substrate including a bioresorbable polymer.

It is preferred that the coating is performed by a method including electrospinning a contrast medium.

Regarding the contrast medium containing a bioresorbable polymer and an iodine component, the above detailed description may be identically applied.

Hereinafter, the bioresorbable stent according to the present invention will be described in detail, referring to what is shown in the drawings.

The bioresorbable stent 1 according to an exemplary embodiment of the present invention includes:
a plurality of rings 10 arranged to be spaced apart at a predetermined interval in the axial direction; and
at least one bridge 20 which is arranged between adjacent two rings of the plurality of rings and connects the adjacent two rings,
wherein in each of the plurality of rings, a wave-shaped unit structure including a protrusion 200 and a depression 100 is arranged in a repeating manner along the circumferential direction,
the unit structure includes the protrusion and the depression asymmetrically, and
the bridge has a curvature.

Herein, the ring 10 is a strut.

More specifically, the ring 10 is most preferably formed of 4 to 8 cells or 6 cells, in which the cell refers to a wave-shaped unit structure including a protrusion 200 and a depression 100. When a ring positioned on one side of each of the bridges is referred to as a first ring and a ring positioned on the other side is referred to as a second ring, one side of each of the bridges is connected to the depression of the first ring and the other side is connected to the protrusion of the second ring, but the one side of each of the bridges may be connected to a position eccentric to one side from the center of the depression of the first ring in the depression of the first ring and the other side may be connected to a position eccentric to the other side from the center of the depression of the second ring in the depression of the second ring. That is, the ring 10 may have an open cell structure connected to three bridges 20. The stent according to an exemplary embodiment of the present invention has an open cell structure, thereby having significantly improved flexibility. In addition, a re-coil change after balloon inflation may be minimized by the bridge. Herein, the center refers to the most depressed portion in the depression and the most protruding portion in the protrusion. The number of rings may be 14 to 18, preferably 16, and there is no phase difference between each ring.

A spacing between the ring 10 and another ring adjacent thereto, that is, a straight distance may be 1.00 mm to 1.3 mm, and a stent substrate having a straight distance of 1.15 mm was manufactured by an exemplary embodiment.

It is preferred that the bridge 20 is manufactured to increase an amount of the bioresorbable polymer by a shape having a curvature, and a bridge having two curvatures was manufactured by an exemplary embodiment. In addition, it is preferred that the bridges intersect.

The stent substrate may have a diameter of about 2.2 mm to 2.8 mm or 2.4 mm to 2.6 mm, preferably 2.503 mm. The strut may have a thickness of about 0.09 mm to 0.13 mm or 0.10 mm to 0.12 mm, preferably 0.11 mm. The strut may have a width of about 0.10 mm to 0.20 mm or 0.13 mm to 0.17 mm, preferably 0.15 mm. The strut may have a surface area of about 30 mm² to 50 mm² or 35 mm² to 40 mm², preferably 37.325 mm².

Since the stent provided in one aspect of the present invention is a bioresorbable stent including a bioresorbable polymer, unlike a conventional metal stent, the material physical properties and the components are different from those of the metal stent. Therefore, a stent design (structure) is very important. Therefore, the stent according to an exemplary embodiment of the present invention may include a spiral cell structure which is effective for a crimping process of the balloon catheter and the bioresorbable stent, and may have an optimized open cell structure when applied to an irregular and tortuous blood vessel. In addition, a bridge having a curvature for minimizing re-coil after balloon inflation is included. Herein, the bridge is connected to an eccentric position in the center of the protrusion and the depression, thereby more effectively minimizing the re-coil change. Therefore, since the stent according to an exemplary embodiment of the present invention has the above structure, it has low foreshortening and high flexibility and radial force, and thus, may be useful for insertion into a blood vessel having a small diameter, acute occlusive lesion, imminent occlusive lesion, and the like. In addition, since radiolucency is increased by coating the contrast medium including iodine on the stent, the stent has high radiography contrast even when a procedure is performed with real-time radiography, and thus, is very efficient.

### MODE FOR CARRYING OUT THE INVENTION

### <Example>Manufacture of Bioresorbable Stent

Step 1: Manufacture of Bioresorbable Stent Substrate

For manufacturing a bioresorbable stent (BRS), a femtosecond laser was used to manufacture a stent shape including poly-L lactic acid (MatWeb— Zeus Absory PLLA bioabsorbable polymer) which is a bioresorbable polymer.

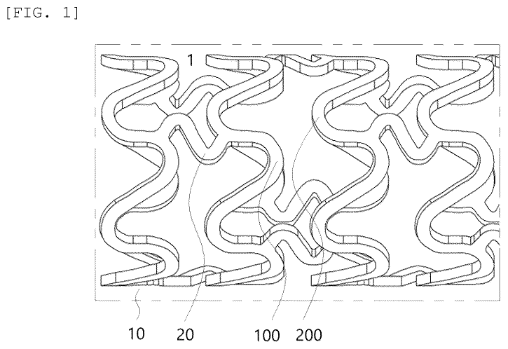

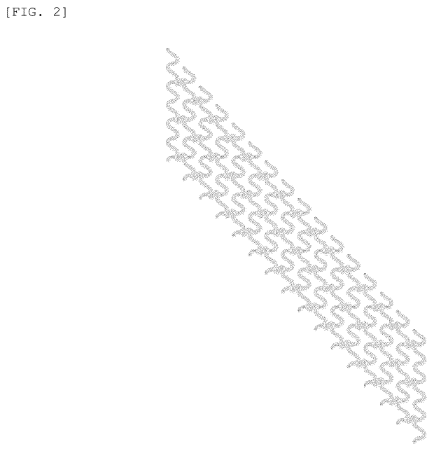

The bioresorbable stent substrate 1 according to an exemplary embodiment of the present invention basically included: a plurality of rings 10 which were arranged to be spaced apart at a predetermined interval in the axial direction; and at least one bridge 20 which was arranged between adjacent two rings of the plurality of rings and connects the adjacent two rings. Each of the plurality of rings had a wave-shaped unit structure including a protrusion 200 and a depression 100 which was arranged in a repeating manner along the circumferential direction (FIGS. 1 and 2).

Step 2: Iodine-Containing Contrast Medium Coating

In order to coat the iodine-containing contrast medium on the bioresorbable stent substrate manufactured in step 1, a vascular contrast medium used in clinical practice (Omnihexol) was filled into a hamilton syringe and coated on the stent substrate manufactured in step 1 using an electrospray system. The coating proceeded at a distance of 60 cm and an angle of 30° under the conditions of a voltage of 10 V and a rotation speed of 50 rpm while a jig moved to the x-axis at a speed of 500 mm/min and a syringe pump was sprayed at 60 μm/min.

### <Experimental Example 1>Finite-Element Analysis

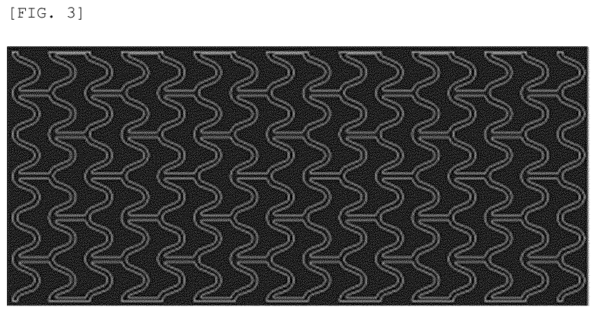

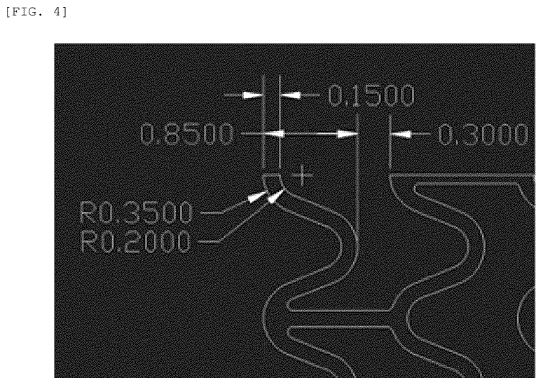

With the finite-element analysis of the bioresorbable stent substrate manufactured in step 1 of Example 1, radial force, foreshortening, crush resistance, and flexibility were tested as follows. As a comparative example, a commonly used, commercialized stent was reversely designed and used. The stent used as the comparative example had a form in which a bridge was positioned parallel to the x-axis in a 6-cell and 16-ring structure, and had a strut width of 0.15 mm, an inner radius of 0.20 mm, an outer radius of 0.35 mm, a ring width of 0.85 mm, a spacing between rings of 0.3 mm, and a surface area of 36.8924 mm² (FIGS. 3 and 4).

1-1. Radial Force

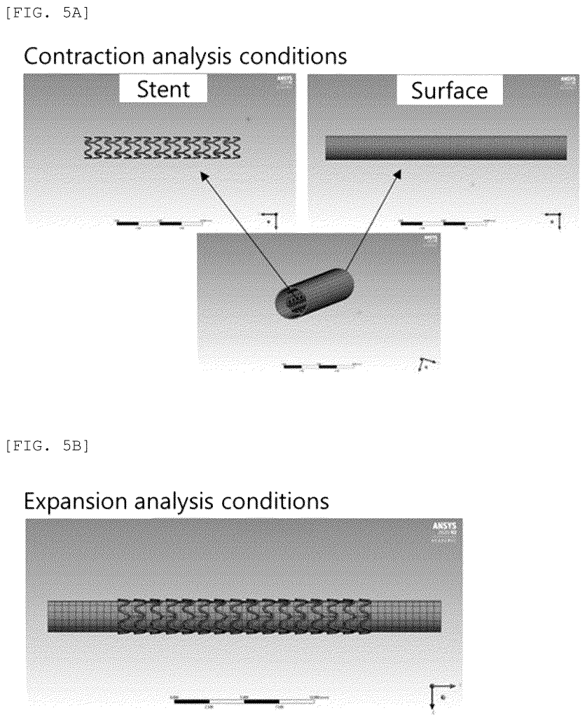

In order to analyze radial force of the example and the comparative example (FIGS. 5A and 5B), a force applied to a blood vessel in the state in which a self-expandable stent was developed during expansion and compression was measured. Specifically, to the stents of the example and the comparative example, thickness and mesh conditions as contraction and expansion analysis conditions were applied, respectively, and then maximum and minimum generated stresses were confirmed. As the contraction analysis conditions, the stents of the example and the comparative example were given a thickness of 0.11 mm, the surfaces were given a thickness of 0.1 mm, and the diameter of the surface positioned outside the stent was shrunk to 1.5 mm. As the expansion analysis conditions, the stents were given a thickness of 0.11 mm, the surfaces were given a thickness of 0.1 mm, and the diameter of the surface positioned inside the stent was expanded to 3 mm. The maximum and minimum stress values upon contraction and expansion of the example and the comparative example as a result are shown in the following Table 1.

TABLE 1

| | Stress upon | | Stress upon | |
|---|---|---|---|---|
| | contraction (MPa) | | expansion (MPa) | |
| | | Comparative | | Comparative |
| | Example | Example | Example | Example |
| Maximum value | 1.9818 | 2.3892 | 1.4655 | 0.54501 |
| Minimum value | 780.34 | 643.75 | 586.92 | 558.32 |
| Average value | 150.2 | 153.13 | 118.53 | 101.71 |

1-2. Foreshortening

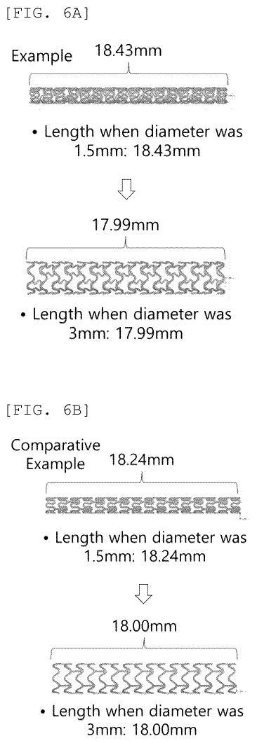

For foreshortening analysis of the example and the comparative example, change in length when each stent was developed to the state attached to the catheter and the indicated value was measured. Specifically, the change in length when the diameter of each stent was 1.5 mm and 3 mm was measured and the results are shown in FIGS. 6A and 6B. In addition, change in length before and after development was compared, foreshortening was calculated therefrom, and the calculated values are shown in the following Table 2.

Foreshortening (%)=[{(length before contraction)−(length after contraction)}/(length before contraction)]×100

TABLE 2

| | Example | Comparative Example |
|---|---|---|
| Foreshortening (%) | 2.38 | 1.31 |

1-3. Crush Resistance

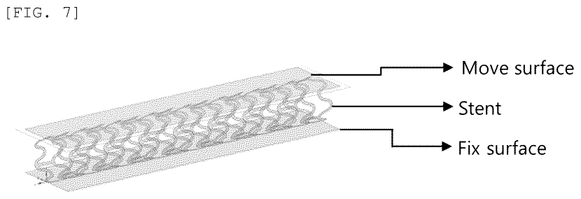

For crush resistance analysis of the example and the comparative example (FIG. 7), a parallel plate was used to determine a load required to cause clinically appropriate buckling or deflection which is equivalent to a decrease in diameter of at least 50% or more and a load required to permanently deform or completely collapse the stent, and it was confirmed whether the stent restored its original shape after the test. As the analysis conditions, the stent was given a thickness of 0.11 mm, a fixed surface and a moving surface were given a thickness of 0.1 mm, and then the moving surface was given a forced displacement by 1.25 mm which was 50% of the stent diameter in the y-axis direction. The maximum and minimum stress values measured as a result are shown in the following Table 3.

TABLE 3

| | Example (MPa) | Comparative Example (MPa) |
|---|---|---|
| Maximum value | 280.73 | 253.11 |
| Minimum value | 0.52636 | 6.3153e−003 |
| Average value | 66.157 | 64.363 |

As a result of analysis, the stent according to the example showed higher crush resistance than the commercially available comparative stent through the stent substrate structure, and thus, it was confirmed to be more appropriate for use as a stent.

1-4. Flexibility

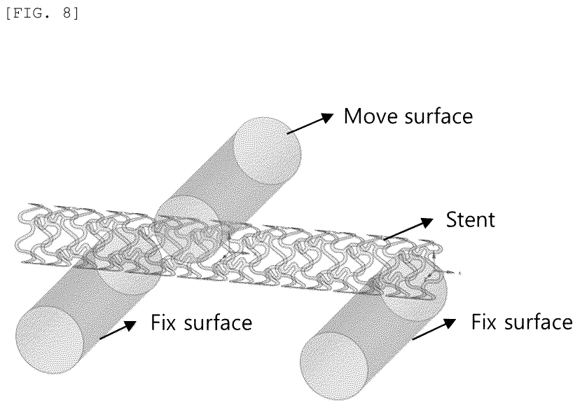

For flexibility analysis of the example and the comparative example (FIG. 8), a minimum radius at which the stent may be bent without twisting or a decrease in diameter of more than 50% was determined, and it was confirmed whether their original shapes were restored after the test. The analysis proceeded referring to a modelling file of Stent flexibility test jig provided by CGblo, and specifically, the stent was given a thickness of 0.11 mm, three stents were given a thickness of 0.1 mm, and the moving surface was moved by 2.2 mm in the y-axis direction to confirm the maximum stress before and after compression.

As a result, the maximum stress value of the comparative example was 0.0079628 MPa, and the maximum stress value of the example was 0.0076613 MPa. Therefore, since the stent having the structure of the stent substrate according to the example may be bent with less force, it has excellent flexibility and thus, is useful for curved blood vessels and allows a convenient procedure.

As a result of predicting the physical property values of the stent structure manufactured in the example by the finite-element analysis, the stent of the example was confirmed to be appropriate for use as a medical stent and the like. Thus, it was confirmed that the real stent of the example showed excellent physical properties by the mechanical test of the following Experimental Example 2.

### <Experimental Example 2>Mechanical Test

With the mechanical test the bioresorbable stent substrate manufactured in step 1 of Example 1, radial force, foreshortening, flexibility, and re-coil were tested as follows.

2-1. Radial Force

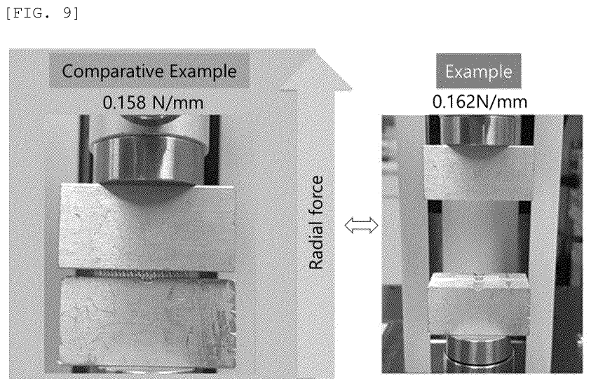

It has been reported that when a stent is inserted into a blood vessel having a small diameter, a chronic total occlusion (CTO), an aorta ostial lesion, a calcified lesion, and the like, a stent having a high radial force is appropriate. The radial force may be confirmed by measuring a force applied to a blood vessel in the state in which the stent is developed during expansion and compression. The radial forces of the example and the comparative example were measured as in FIG. 9, and the results are shown.

As a result, as confirmed in FIG. 9, the radial force of a control stent was 0.158 N/mm and the radial force of the stent of the example was 0.162 N/mm, and since the stent according to the example of the present invention had a higher radial force than the commercially available stent, it would be more appropriate to use the stent of the example for insertion into a blood vessel having a small diameter, a chronic total occlusion, or the like.

2-2. Foreshortening

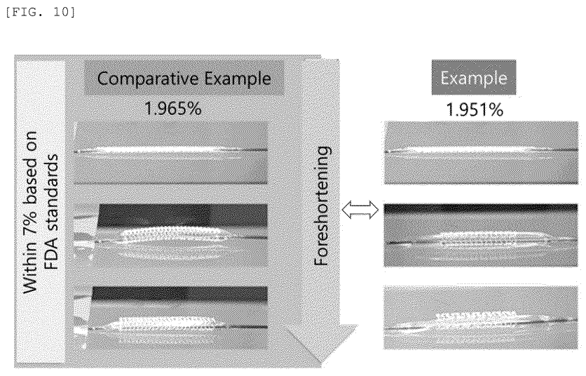

The foreshortening may be confirmed by measuring a change in length when the stent is developed to the state of being mounted on the catheter and the indicated value. In order to use the stent as a stent for cardiovascular system, a stent having no change in foreshortening before and after expansion when the stent was developed to the state of being mounted on a balloon catheter and the indicated value is appropriate. The foreshortenings of the example and the comparative example were measured as in FIG. 10, and the results are shown.

As a result, as confirmed in FIG. 10, the foreshortening before and after development of the comparative example was 1.965% and the foreshortening before and after development of the example was 1.951%, both of which were low values, and in particular, it is shown that the stent of the example had a less change in length before and after development than the commercialized stent of the comparative example.

2-3. Flexibility

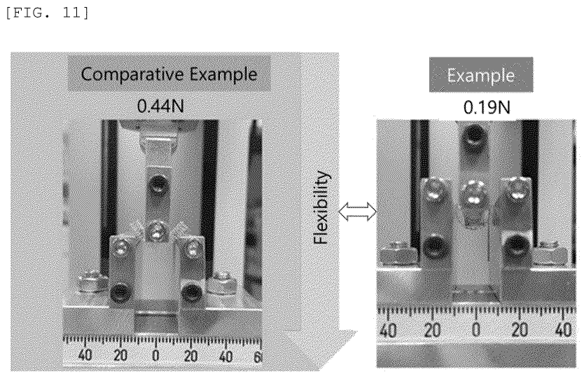

It is appropriate to use a stent having excellent flexibility in a blood vessel having a small diameter of 3 mm or less, acute occlusive and imminent occlusive lesions, proximal tortuosity, and an acute angulation lesion at 45° or more. Bending/twisting/flexibility of the stent may be confirmed by determining a minimum radius at which the developed stent is bendable without twisting or a decrease in radius of more than 50% and seeing whether its original shape is restored after the test. The flexibilities of the example and the comparative example were measured as in FIG. 11, and the results are shown.

As a result, as confirmed in FIG. 11, the stent of the example had better flexibility than the stent of the comparative example and was bendable with less force, and thus, may be useful for curved blood vessels.

2-4. Re-Coil

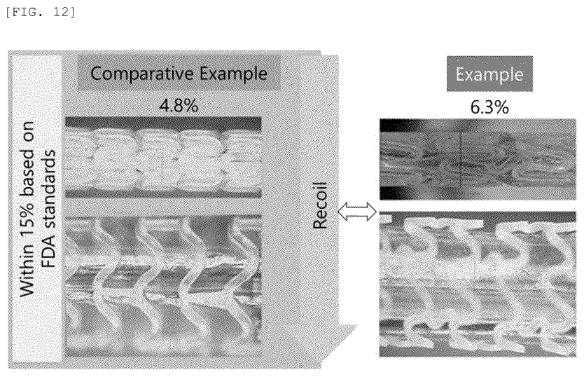

The re-coil of the stent may be confirmed by determining the amount of re-coil after developing a balloon expansion stent in the state in which there is no internal load in order to determine a stent diameter in a developed state. A stent having no change in re-coil before and after expansion when it is developed to the state of being mounted on a balloon catheter and the indicated value is appropriate for use as a cardiovascular stent and the like. The results of confirming the re-coils of the stents of the example and the comparative example are shown in FIG. 12.

As a result, a change in re-coil of the comparative example was smaller than that of the example, but since the stent of the example met the FDA standards (within 15%), it was confirmed to have no problem in clinical use.

### <Experimental Example 3>Analysis Before and After Contrast Medium Coating

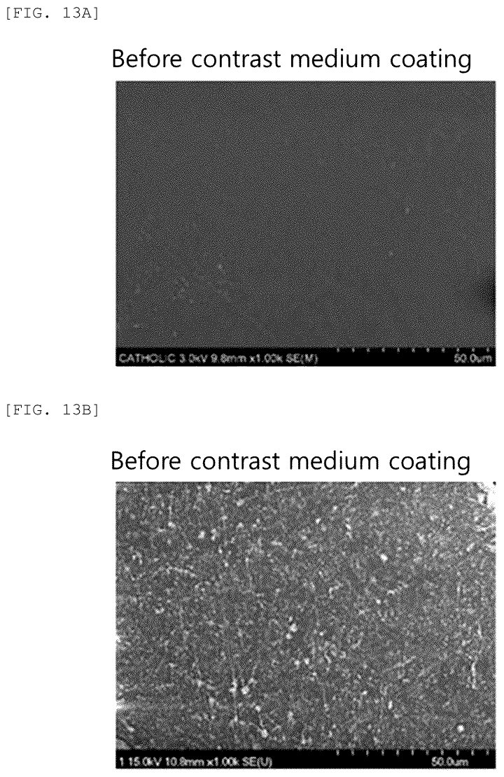

Elemental analysis (EDX, Energy-Dispersive X-ray spectroscopy) and surface scanning electron microscope (SEM) analysis were performed before and after coating the contrast medium containing iodine according to step 2 of the example and the results are shown in the following Table 4 and FIGS. 13A and 13B. As a result of the elemental analysis, it was confirmed that there were only C and O elements before contrast medium coating, but about 54% of an I element existed after contrast medium coating.

TABLE 4

| | Before contrast medium coating | | After contrast medium coating | |
|---|---|---|---|---|
| | weight % | atomic % | weight % | atomic % |
| CK | 52.5 | 59.55 | 29.25 | 62.29 |
| O | 47.5 | 40.45 | 16.78 | 26.83 |
| IL | 0 | 0 | 53.97 | 10.88 |
| Totals | 100 | 100 | 100 | 100 |

### <Experimental Example 4>Radiolucency Analysis

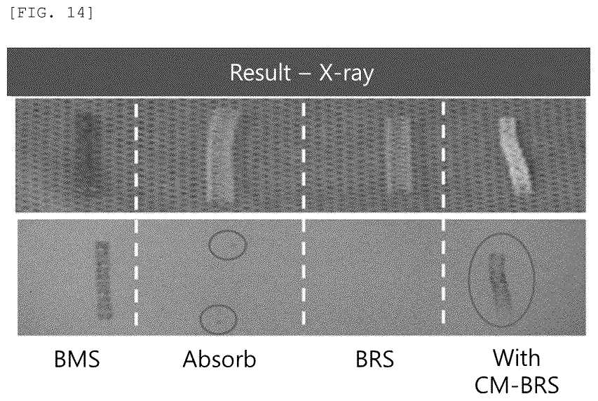

X-ray analysis (BV PULSERA, PHILIPS) was performed for radiolucency analysis of a bioresorbable stent before contrast medium coating (BRS) manufactured in step 1 of the example, a bioresorbable stent after contrast medium coating (with CM-BRS) manufactured in step 2, a commercialized stent (Absorb), and a metal stent (BMS). The results are shown in FIG. 14.

As a result, since the metal BMS was formed of metal, its radiopacity was high and the metal BMS was clearly shown in the X-ray image. Since Absorb was formed of a polymer and had a metal marker made of Pt—Cr at both ends, the polymer was all radiolucent and was not shown in the X-ray and only Pt—Cr at both ends was shown. Since BRS is made of a polymer, it was radiolucent and was not shown in the X-ray image. With CM-BRS was clearly shown in the X-ray image. Thus, it was confirmed that by coating the contrast medium on the bioresorbable stent, the radiopacity was improved to improve radiography contrast.

Hereinabove, though the present invention has been described in detail by the preferred examples and experimental examples, the scope of the present invention is not limited to specific examples, and should be construed by the appended claims. In addition, it should be understood by a person skilled in the art that many modifications and variations are possible without departing from the scope of the present invention.

### Detailed Description of Main Elements

1: Stent substrate
10: Ring
20: Bridge
100: Depression
200: Protrusion

1. A bioresorbable stent comprising:
a stent substrate including a bioresorbable polymer; and
a contrast medium containing an iodine component, coated on the stent substrate.

2. The bioresorbable stent of claim 1, wherein the bioresorbable polymer is one or more selected from the group consisting of polylactic acid, polylactide, polyglycolide, polycaprolactone, polylactide-co-glycolide, polylactide-co-caprolactone, polyglycolide-co-caprolactone, polydioxanone, polytrimethylene carbonate, polyglycolide-co-dioxanone, polyamide ester, polypeptide, polyorthoester-based, polymaleic acid, polyphosphagen, polyanhydride, polysebacic anhydride, polyhydroxyalkanoate, polyhydroxybutylate, or polycyanoacrylate.

3. The bioresorbable stent of claim 1, wherein the bioresorbable polymer includes poly-L-lactic acid.

4. The bioresorbable stent of claim 1, wherein the contrast medium containing an iodine component increases radiopacity.

5. The bioresorbable stent of claim 1, wherein the contrast medium containing an iodine component includes a contrast medium selected from the group consisting of Iopromide, Iopamidol, Iohexol, Iodixanol, Amidtrizoic acid, Iokisagulic acid, Ioxylan, Iotalamic acid, Isotroxylic acid meglumine, Iotrolan, Iopanoic acid, Iomeprol, sodium iofordate, Iodamide, iodochisamic acid, and combinations thereof.

6. A method for manufacturing a bioresorbable stent, the method comprising: coating a contrast medium containing an iodine component on the stent substrate including a bioresorbable polymer.

7. The method for manufacturing a bioresorbable stent of claim 6, wherein the coating includes electrospinning the contrast medium.

8. A bioresorbable stent comprising:
a plurality of rings which are arranged to be spaced apart at a predetermined interval in the axial direction; and
at least one bridge which is arranged between adjacent two rings of the plurality of rings and connects the adjacent two rings,
wherein in each of the plurality of rings, a wave-shaped unit structure including a protrusion and a depression is arranged in a repeating manner along the circumferential direction,
the unit structure includes the protrusion and the depression asymmetrically, and
the bridge has a curvature.

9. The bioresorbable stent of claim 8, wherein when a ring positioned on one side of each of the bridges is referred to as a first ring and a ring positioned on the other side is referred to as a second ring,
one side of each of the bridges is connected to the depression of the first ring and the other side is connected to the protrusion of the second ring,
but the one side of each of the bridges is connected to a position eccentric to one side from the center of the depression of the first ring in the depression of the first ring and the other side is connected to a position eccentric to the other side from the center of the depression of the second ring in the depression of the second ring.

10. The bioresorbable stent of claim 8,
wherein the number of rings is 14 to 18, and
a straight distance between the adjacent rings is 1.00 mm to 1.3 mm.

11. The bioresorbable stent of claim 8, wherein the bridge has two curvatures.
